(19) United States
(12) Patent Application Publication
(10) Pub. No.: US 2008/0195173 A1
(43) Pub. Date: Aug. 14, 2008

(54) Method and Apparatus for Treating Sleep Apnea and Snoring

(75) Inventor: Franco Ugo Villa, Stouffville (CA)

Correspondence Address: INTEGRAL INTELLECTUAL PROPERTY INC., 1370 DON MILLS ROAD, SUITE 300, TORONTO, ON, M3B 3N7, omitted

(73) Assignee: CANZZZ INVESTMENTS LTD., Mississauga (CA)

(21) Appl. No.: 11/674,780

(22) Filed: Feb. 14, 2007

(51) Int. Cl. A61N 1/00

(52) U.S. Cl. 607/42

(57) ABSTRACT

A method and device for treating snoring and obstructive sleep apnea, in which one sensor senses the onset of an episode of snoring or sleep apnea and causes production of an electrical stimulating signal consisting of a combination of direct current and a train of alternating pulses, with the respective functions of opening up restricted airway passages by stimulation of related muscle groups and eliciting a response of partial arousal, in which the condition is corrected. The onset of the episodes is sensed by an acoustic transducer, the episodes being differentiated by their unique acoustical signature by means of appropriate signal processing, the acoustical transducer also carrying one of two stimulation electrodes, which are held in light contact against the throat of the subject by means of a lightweight neckband.

### TECHNICAL FIELD

Embodiments of the present invention relate to an apparatus for treating snoring and obstructive sleep apnea, or either of them, and also relate to a method of treatment of these conditions, using electrical stimulation.

### BACKGROUND

Snoring is a condition commonly caused by a partial obstruction of the upper airway, in which the airflow causes the obstructing tissue to resonate, resulting in characteristic and well known snoring sounds. Snoring can be a major disruptive agent in relationships where one person is subjected to sleep disturbing effects, which the snoring partner causes.

Obstructive sleep apnea is a more serious medical condition caused by a more pronounced obstruction such that the airflow during breathing is severely restricted or is completely interrupted. This condition causes a degraded sleep pattern and reduced blood oxygenation and is believed to have the potential for producing serious medical problems, including cardiovascular complications.

Various solutions are well known for treating upper airway obstructions. Some of these solutions involve an insertion of mechanical devices, or even surgical interventions. Subjecting the airway to a constant pressure of a few millibars by means of a mask connected to a pressurized air source (a technique known as Continuous Positive Airway Pressure) is a proven method of controlling sleep apnea. However, these techniques are invasive and therefore persons are reluctant to use them.

Other less invasive techniques are known in which the onset of the obstructive condition is detected and a stimulus is applied to disturb the person, in order to elicit a response in which the condition is corrected. Examples of devices and techniques belonging to this class are shown in U.S. Pat. Nos. 3,480,010 and 4,715,367 issued to Crossley; U.S. Pat. No. 3,696,377 issued to Wall; U.S. Pat. No. 4,220,142 issued to Rosen et al.; U.S. Pat. No. 4,593,686 issued to Lloyd et al.; U.S. Pat. Nos. 6,666,830 and 6,935,335 issued to Lehrman et al. and U.S. Pat. No. 6,371,120 issued to Chiu et al. These devices operate by a variety of techniques which have in common two aspects; firstly, detection of the obstructed airway condition (either snoring or sleep apnea), and secondly, applying a stimulus to the subject in respect to such detection.

Recent research has shown that obstructions in upper airways may be cleared with electrical stimulation. Two articles describing this research may be found in American Review of Respiratory Disease, Vol. 140, 1989 at pp. 1279-1289. The first article is entitled “Effects of Electrical Stimulation of the Genioglossus on Upper Airway Resistance in Anesthetized Dogs” by Hiroshi Miki et al. The second article is entitled “Effects of Submental Electrical Stimulation During Sleep on Upper Airway Patency in Patient with Obstructive Sleep Apnea” also by Hiroshi Miki et al. U.S. Pat. No. 4,830,008 issued to Meer discusses an implantable system for treatment obstructive sleep apnea by means of electrical stimulation. A system achieving the same goal by the same means without resorting to implantation is discussed in U.S. Pat. No. 5,265,624 issued to Bowman. In this system electrodes are placed on the gums, thus in the immediate vicinity of the genioglossus and related muscle groups in the upper airway, by means of a mouthpiece fitted with passive circuitry energized by radio frequency emitted by a collar assembly which incorporates the power supply and event detector. Radio frequency is processed within the mouthpiece in order to power the circuit and issue a train of narrow unidirectional pulses to the stimulating electrodes. Although not made evident in the writing, unidirectional pulses are effectively integrated into a direct current by storage components of capacitive and electrolytic nature intrinsic of the circuit pathway. Thus Bowman also relies on DC stimulation of the muscles of interest.

A disadvantage of the prior art devices is that there is a substantial likelihood that the stimulus they apply to the subject will degrade the subject's sleep quality. This negates the primary objective of restoring a restful sleep to the subject. Also, subjects are deterred from using user unfriendly, cumbersome contrivances such as that proposed by Bowman.

### SUMMARY

An apparatus for treating an obstruction of the upper airway of a person, such condition causing an episode of either snoring or obstructive sleep apnea, comprises:

a) a sensor for sensing the onset of said episode;

b) a stimulus generator coupled to said sensor and responsible thereto for generating a stimulation signal for application to said person, said signal comprising a direct current and a pulsating current;

c) said generator including a circuit activated upon the sensing of said episode for regulating the relative levels of said direct current and of said pulsating current as said stimulation signal is applied to said person; and

d) a sensor for detecting the end of said episode and being coupled to said generator for terminating the application of said stimulation signal to said person following the end of said episode.

A method of treating a person having an obstruction of the upper airway causing the onset of either a snoring or an obstructive sleep apnea episode comprises:

a) sensing the onset of said episode;

b) upon the detection of said onset, applying an electrical stimulation signal to said person, said signal comprising a direct current and a pulsating current;

c) regulating the relative levels of said direct current and said pulsating current as said signal is applied to said person;

d) sensing termination of said episode; and

e) following sensing said termination, terminating the application of said stimulation signal to said person.

### BRIEF DESCRIPTION OF THE DRAWINGS

Embodiments are illustrated by way of example and without limitation in the figures of the accompanying drawings, in which like reference numerals indicate corresponding, analogous or similar elements, and in which:

FIG. 1 is a plan view of the inner front part of a neck strap, which supports components of a system according to the invention;

FIG. 2 is a side sectional view of an electroacoustic transducer;

FIG. 3 is a block diagram of a detection circuit which detects snoring and sleep apnea episodes; and

FIG. 4 is a block diagram of a stimulus generator.

### DETAILED DESCRIPTION

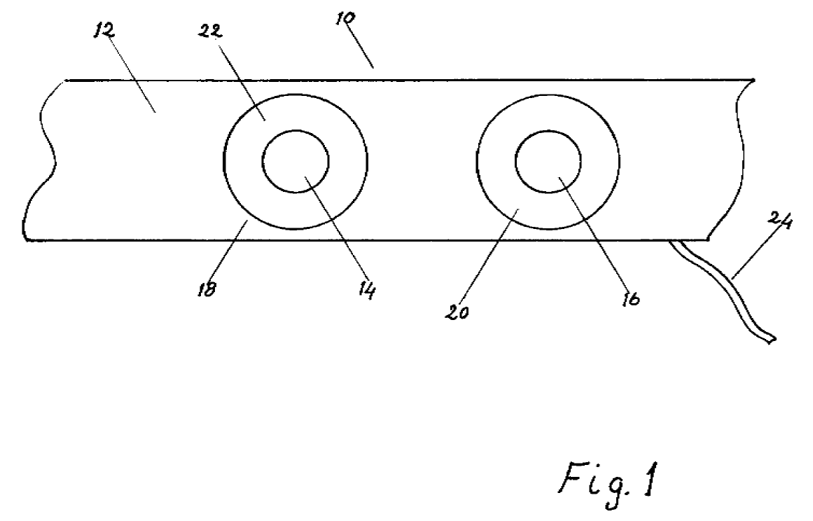

Reference is made to FIG. 1, which is a plan view of the inner front of a neck strap 10 for use with embodiments of the present invention. The neck strap 10 includes a main substrate 12 of woven material with a sufficiently open weave to allow for evaporation of skin moisture. The neck strap 10 supports conductive stimulation pads 14, 16 in spaced apart relation, an electroacoustic transducer 18 comprising an acoustic sensing diaphragm 22 and a conductive stimulation pad 14, and an insulating disc 20 which carries conductive stimulation pad 16. The output of transducer 18 and conductive stimulation pads 14, 16 are connected to the supporting circuits for use with embodiments of the present invention by means of cable 24. Cable 24 also carries stimulation signals to conductive stimulation pads 14 and 16. The neck strap 10 does not need to apply more than a gentle pressure on the skin, since the stimulation circuit path is of high impedance value at the source, to minimize the effects of such variables as resistance at the pad-skin interface and others of physiological nature.

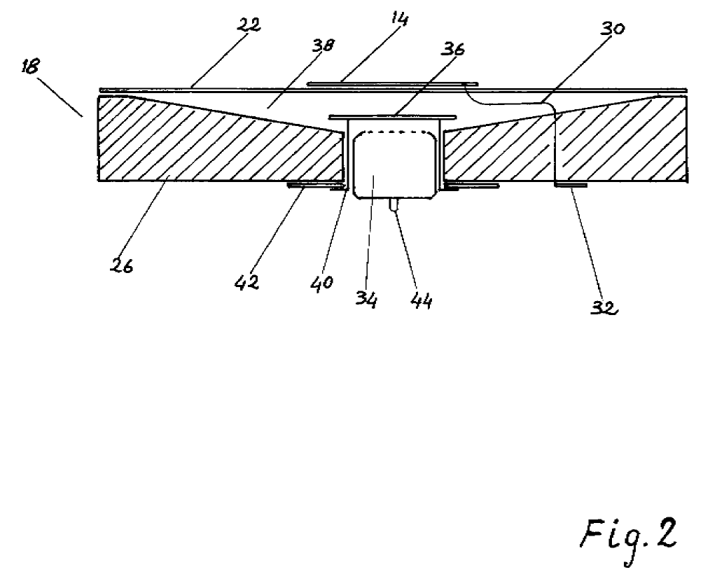

FIG. 2 is a side section view of electroacoustic transducer 18, which includes supporting structure 26 made of insulating plastic such as polycarbonate. The front of transducer 18 carries acoustic sensing diaphragm 22 made of thin polyamide foil or other material with similar mechanical electrical specifications. Diaphragm 22 is bonded at the periphery to the flat rim of supporting structure 26 and it carries conductive stimulation pad 14, pad 14 being in the form of a disc made of thin stainless steel foil bonded to diaphragm 22 at the centre of diaphragm 22. A thin conductive wire 30 connects pad 14 to terminal 32 on the back of the supporting structure 26. Air cavity 38 is the site where an acoustic pressure is generated by diaphragm 22 when diaphragm 22 is vibrating in response to acoustical events such as breathing airflow and snoring, by virtue of being positioned in intimate contact with the skin of the throat. The acoustic pressure is converted into an electrical signal by microphone 34, which is affixed with a sealing bond in a centre hole of supporting structure 26. A shielding disc 36 is an electrostatic shield which prevents stimulation pulses present on pad 14 from interfering with the minute signal output by microphone 34. Shielding disc 36 is mechanically supported by three or four wires 40 which have also electrically connect disc 36 to ground ring 42 which is the ground reference of the circuit. This connection further aids in making the output of microphone 34 free of interference. Terminal 44 is the output connection of microphone 34.

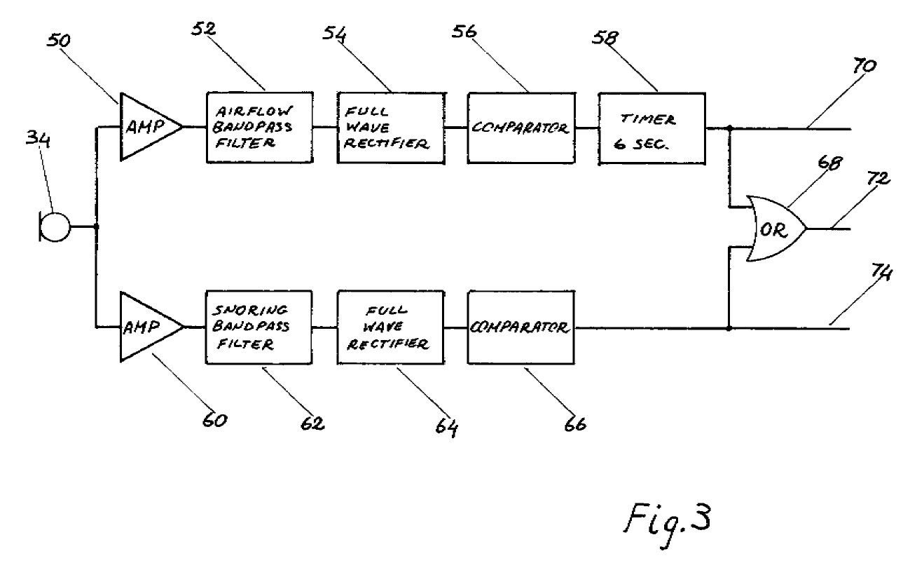

FIG. 3 is a block diagram of a detection circuit which detects snoring and sleep apnea episodes. In the detection circuit the signal from microphone 34 is amplified by amplifiers 50, 60 and then applied in two branches to bandpass filters 52, 62 which extract telltale signatures respectively of breathing and snoring activity. Various types of signal processing are well known to those skilled in the art to detect breathing activity and snoring activity and to filter out extraneous signal components. The snoring signal is directed to full wave rectifier 64 and then to internally referenced comparator 66, which outputs a logic one if snoring is sensed.

The breathing signal from bandpass filter 52 is rectified by full wave rectifier 54, the output of which is directed to internally referenced comparator 56 which outputs a logic one if the acoustic signature of air flow is detected. The output of comparator 56 is used to reset a timer 58, the output of which is set to go high after an interval of four to six seconds following any reset. Respiration cycles are typically four to six seconds and therefore the timer 58 is reset every equivalent time interval (i.e. every respiration cycle). Thus the timer 58 will output a logic one if a breathing pattern is not detected for more than six seconds. The range of four to six seconds is suggested as from experimentation this appears to be the optimal range for effective use of embodiments of the present invention. However it is not the intent of the inventor to restrict embodiments of the invention to this range, other ranges may be utilized if found to be effective.

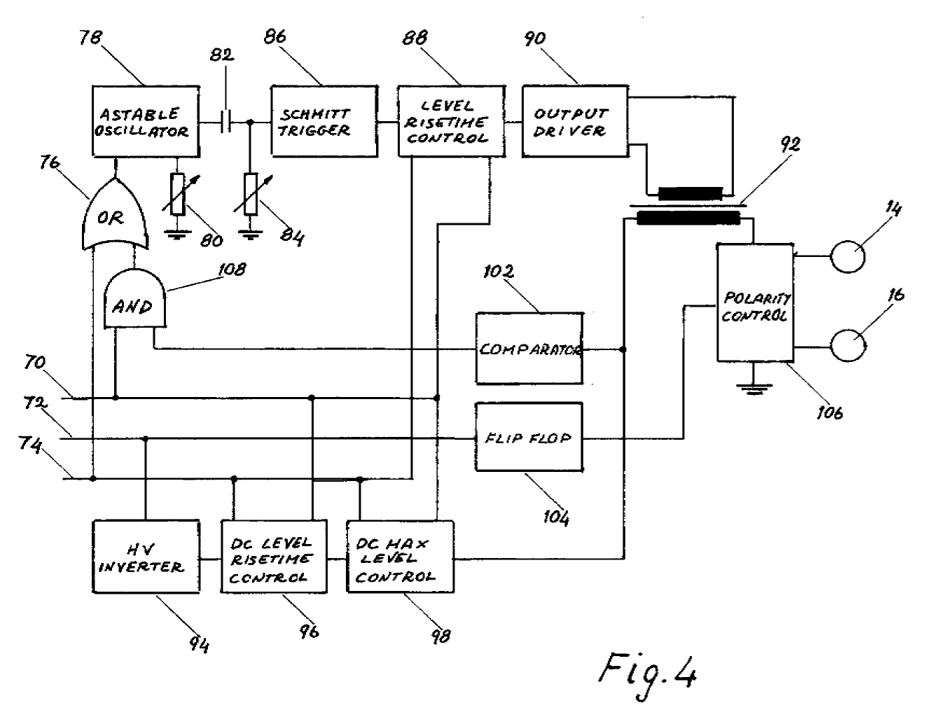

Snoring events are characterized by a logic one at the output 74 and apnea events by a logic one at output 70. Outputs 70, 74 are directed to the inputs of OR gate 68, which outputs at 72. The information present at points 70, 72, 74 is directed to the processing circuits of the stimulus generator illustrated with reference to FIG. 4.

FIG. 4 is a block diagram of a stimulus generator. Astable oscillator 78 is set running by a logic one at the output of OR gate 76. The frequency of astable oscillator 78 is controlled by means of variable resistor 80 within a range of approximately three to 10 pulses per second. The output of astable oscillator 78 is directed to a Schmitt trigger 86, via a resistor-capacitor network of capacitor 82 and variable resistor 84, with a time constant in the range of fifty microseconds to fifty milliseconds. The output of Schmitt trigger 86 is a train of pulses with a width equivalent to the time constant of the input network. The pulse train issued by Schmitt trigger 86 is directed to level risetime control 88, which sets the amplitude of the pulses at a pre-selected value. On one embodiment the time for pulse amplitude to reach the pre-selected value is made to be within 1 second after the sensing of a snoring event and seconds following the sensing of an apnea event, the information being supplied by apnea and snoring information present at points 70 and 74 respectively. The output of level risetime control 88 is directed to output driver 90, which also converts the unidirectional pulses at its input into symmetrical square waves, which are then applied to the primary of step-up transformer 92. Thus square waves with the required high voltage are present at the secondary winding of transformer 92.

HV inverter 94 raises the power source voltage to the level required for stimulation. As one skilled in the art can appreciate any number of power sources may be used to power the circuitry of embodiments of the present invention, a battery being one such example. The time for the voltage to reach a maximum value is set by DC level risetime control 96. In one embodiment the time would be three seconds after the onset of an apnea event and zero seconds after the onset of snoring, this being accomplished by pertinent information at points 70, 74 applied to level risetime control 96. Maximum level is set by DC max level control 98 which in one embodiment reduces the level by 50% if snoring information is present at point 74.

DC at the output of level control 98 is directed to polarity control unit 106 via the secondary of transformer 92, which is connected in series in the stimulation circuit. A stimulation signal thus consists of a DC current with a square wave pulse train superimposed on it, the balance between the two modes being apt to be adjusted as required. This composite signal is directed to conductive stimulation pads 14, 16 via polarity control 106, which assigns ground level and high level to either one depending on the output status of FLIP-FLOP 104 which is clocked by apnea or snoring information at point 72. Thus the polarity applied to conductive stimulation pads 14, 16 is reversed at every new episode of stimulus generator operation, this being done to counteract effects of a constant polarity DC current such as electrolytic effects at the pad-skin interface and possible increase of the threshold of stimulation level.

Following the onset of a snoring episode, characterized by a logic one at point 74 which starts astable oscillator 78 by means of OR gate 76, pulse stimulation is applied to the subject with the level reaching maximum in one second, the level rise being interrupted at the point which causes the event to cease. Following the onset of a snoring episode DC stimulation is also applied to the subject, with a level reaching 50% of a pre-set maximum with no delay. DC stimulation is also interrupted at a level where the condition is interrupted. Reason for such process is that while DC stimulation acts to open the airway with a minimum of perceived effect by the subject, pulse stimulation is perceived as a tingle evoking a condition in which the event is terminated. Said perceived stimulus has the effect of conditioning the subject into subconsciously avoiding a posture conducive to snoring. The sudden application of a reduced DC level also contributes to perception.

Following the onset of an apnea episode, characterized by a logic one at point 70 which activates HV inverter 94, DC stimulation is applied to the subject, the level of which reaches a pre-selected maximum value in three seconds after the onset of the episode. Stimulation is interrupted at the level which causes the event to cease. If the episode is not corrected at maximum DC stimulation level, which is characterized by a logic one at the output of internally referenced comparator 102 and presented to one input of AND gate 108, the other input seeing also logic one at point 70. Therefore the output of gate 108 starts astable oscillator 78, the end result being pulse stimulation with a level risetime of six seconds compounding the DC stimulus.

The conductive stimulation pads 14, 16 through which the stimulation is applied have in one embodiment an active surface of one cm² to five cm² and are positioned approximately five cm to ten cm apart symmetrically to the muscle group to be stimulated. This has been found to also be an optimum area for acoustic detection of the acoustical signature of breathing, being the region of highest turbulence in the air flow, caused by restrictions which lead to or cause snoring and apnea. However, these parameters are not critical. The system is tolerant of axial offset between the stimulated region and the position of the pads, although the best results have been found if the offset is made equivalent to the spacing between the pads.

While the stimulation pulses applied to the subject person may be relatively high voltage, the short duration of the pulses ensures that the overall integrated energy of each pulse is limited to values which are a fraction of a millijoule. Also, the DC stimulation current is limited to fractions of a milliamp, even if the voltages at play may be substantial by reason of a source resistance which is designed to be one order of magnitude or more higher than the combined electrical resistance of skin-pad interface and underlying tissue. The high surface area of the pads 14, 16 ensure that the current density at skin level be kept sufficiently low to avoid disturbing the subject person with the perception of an electric shock.

The neck strap 10 may be fastened by any desired means, e.g. hook and loop pads (not shown) attached to the ends of the strap. The supporting electronics may be housed in a unit smaller than a typical cell phone, placed inside a suitable pocket of a T-shirt or other type of garment. The electrical connection to components on the neck strap is accomplished by a flexible miniature cable (Feature 24 of FIG. 1) in which the conductors carry the microphone signal and stimulation current.

It will be realized that various changes may be made from the preferred embodiments described. For example, separate physical sensors may be used, one to sense the onset of an episode and a different one to sense the termination of the episode. For example, the device of embodiments of the present invention may be placed on a night table or other suitable place or, with suitable miniaturization be contained on the neck strap. Functions of the device may be augmented with snoring and sleep apnea event counters. Different processing means may be used, reflecting continuous, rapid advancements in the electronic arts. The circuits described in the examples shown are examples of one implementation of embodiments of the present invention. As one skilled in the art will recognize the circuits may be created from numerous combinations of discrete components, both analog and digital. Further one skilled in the art can design circuits in other topologies to achieve the desired results. Various muscles or muscle groups may be stimulated to terminate snoring or a sleep apnea episode. Further changes may be made without departing from the scope of the present invention.

I claim:

1. An apparatus for treating an obstruction of the upper airway of a person, such condition causing an episode of either snoring or obstructive sleep apnea, said apparatus comprising:
a) a first sensor for sensing the onset of said episode;
b) a stimulus generator coupled to said first sensor and responsible thereto for generating a stimulation signal for application to said person, said signal comprising a direct current and a pulsating current;
c) said generator including a circuit activated upon the sensing of said episode for regulating the relative levels of said direct current and of said pulsating current as said stimulation signal is applied to said person; and
d) a second sensor for detecting the end of said episode and being coupled to said generator for terminating the application of said stimulation signal to said person following the end of said episode.

2. The apparatus of claim 1, in which said stimulation signal is applied to the throat region of said person by two electrically conductive stimulation pads one for said direct current and the other for said pulsating current.

3. The apparatus of claim 2, further comprising a polarity control for reversing polarity to said electrically conductive stimulation pads for each episode.

4. The apparatus of claim 1, in which said first sensor senses the onset of said episode by sensing a vibration signal pertaining to said episode.

5. The apparatus of claim 4, in which said vibration signal is sensed by means of a diaphragm placed in contact with the skin of the throat of said person.

6. The apparatus according to claim 5, in which said diaphragm is acoustically coupled to a microphone through an air cavity.

7. The apparatus of claim 5, in which an electrically conductive stimulation pad in the shape of a thin electrically conductive disc is bonded to said diaphragm.

8. The apparatus of claim 7, in which a shielding disc is located within said air cavity between said conductive pad and said microphone.

9. An apparatus for treating an obstruction of the upper airway of a person, such condition causing an episode of either snoring or obstructive sleep apnea, said apparatus comprising:
a) a sensor for sensing the onset of said episode and for detecting the end of said episode;
b) a stimulus generator coupled to said sensor and responsible thereto for generating a stimulation signal for application to said person, said signal comprising a direct current and a pulsating current; and
c) said generator including a circuit activated upon the sensing of said episode for regulating the relative levels of said direct current and of said pulsating current as said stimulation signal is applied to said person,
wherein said sensor is coupled to said generator for terminating the application of said stimulation signal to said person following the end of said episode.

10. The apparatus of claim 9, in which said stimulation signal is applied to the throat region of said person by two electrically conductive stimulation pads one for said direct current and the other for said pulsating current.

11. The apparatus of claim 10, further comprising a polarity control for reversing polarity to said electrically conductive stimulation pads for each episode.

12. The apparatus of claim 9, in which said sensor senses the onset of said episode by sensing a vibration signal pertaining to said episode.

13. The apparatus of claim 12, in which said vibration signal is sensed by means of a diaphragm placed in contact with the skin of the throat of said person.

14. The apparatus according to claim 13, in which said diaphragm is acoustically coupled to a microphone through an air cavity.

15. The apparatus of claim 13, in which an electrically conductive stimulation pad in the shape of a thin electrically conductive disc is bonded to said diaphragm.

16. The apparatus of claim 15, in which a shielding disc is located within said air cavity between said conductive pad and said microphone.

17. A method of treating a person having an obstruction of the upper airway causing the onset of either a snoring or an obstructive sleep apnea episode comprising:
a) sensing the onset of said episode;
b) upon the detection of said onset, applying an electrical stimulation signal to said person, said signal comprising a direct current and a pulsating current;
c) regulating the relative levels of said direct current and said pulsating current as said signal is applied to said person;
d) sensing termination of said episode; and
e) following sensing said termination, terminating the application of said stimulation signal to said person.

18. The method of claim 17, wherein said direct current produces an opening of the upper airway of said person by stimulating related muscle groups.

19. The method of claim 17, wherein said pulsating current produces a perception of tingling in said person.

20. The method of claim 17, wherein said direct current and said pulsating current interact to produce a cessation of said episode.

21. The method of claim 17, wherein polarity of the electronic stimulation signal is reversed for each episode.
